(19) United States
(12) Patent Application Publication
(10) Pub. No.: US 2024/0269385 A1
(43) Pub. Date: Aug. 15, 2024

(54) APPARATUS FOR FILLING A FLUID DELIVERY

(71) Applicant: Becton, Dickinson and Company, Franklin Lakes, NJ (US)

(72) Inventors: Dana COTE, Boxford, MA (US); J. Richard GYORY, Sudbury, MA (US); Mark WOOD, Sterling, MA (US); Nicholas ANDERSON, Whitman, MA (US)

(73) Assignee: Becton, Dickinson and Company, Franklin Lakes, NJ (US)

(21) Appl. No.: 18/565,890

(22) PCT Filed: Jun. 7, 2022
(86) PCT No.: PCT/US2022/032501

§ 371 Date: Nov. 30, 2023

Related U.S. Application Data
(60) Provisional application No. 63/209,821, filed on Jun. 11, 2021.

(51) Int. Cl. A61M 5/178; A61M 5/142

(52) U.S. Cl. CPC A61M 5/1782; A61M 2005/14252; A61M 2209/045

(57) ABSTRACT

A fluid delivery device (110) comprising an integrated vial connector system (180) disposed in a fluid path (124) between a reservoir (122) and an insertion mechanism (116). Additionally, a fluid delivery device (210) and an external fill device (294) wherein when the external fill device (294) engages the fluid delivery device (210), a main drive gear (234) is disengaged and a leadscrew (236) is engaged for filling the fluid delivery device (210) with the medicament. Also, a vial adapter (280) comprising an elastomer (282) that engages an outer diameter of a cannula (217) in an insertion mechanism (216) to provide a sealing surface, wherein the vial adapter (280) engages the fluid delivery device (210) and the vial (202) to fill the fluid delivery device (210) with medicament while maintaining a sealed interface. In addition, a vial adapter (380) for engaging a filling station (284) to fill a fluid delivery device (210).

### CROSS-REFERENCE TO RELATED APPLICATION

This application claims priority under 35 U.S.C. 119(e) to U.S. provisional application Ser. No. 63/209,821, filed on Jun. 11, 2021, which is incorporated herein by reference in its entirety

### FIELD OF THE INVENTION

Illustrative embodiments generally relate to pump mechanisms for use in fluid delivery devices such as wearable medication infusion patches. More specifically, illustrative embodiments generally relate to filling mechanisms or methods of filling the fluid delivery device that are integral and external to the fluid delivery device.

### DESCRIPTION OF RELATED ART

Typical drug delivery patch pump designs are challenged by filling a fluid delivery device having a fluid displacement module that is largely rigid and difficult to access. Thus, an innovative mechanism and method is desired to effectively fill the fluid delivery device with medicament.

### SUMMARY

There are no manual filling options available to infusion pump designs with a fixed plunger rigidly attached to a lead screw. A fixed plunger design or rigid attachment of the plunger means that there are no moving parts (rotational or off axis movement) except for axial or translational movement of the plunger connected to a plunger head and leadscrew. Other automated filling options require extra devices to be used for filling. The illustrative embodiments of the present disclosure can provide technical solutions to the above and other technical problems, and additional advantages are realized by illustrative embodiments.

Exemplary embodiments of the present disclosure realize several advantages such as integrating a filling mechanism into the fluid delivery device such as an infusion pump, patch pump and infusion patch. In other words, all filling tools are disposed in the fluid delivery device such that no further devices or components are needed to transport and carry with the fluid delivery device. In other words, the fluid delivery device houses all connections, components and other devices used for filing. Moreover, a motor of the fluid delivery device advantageously incorporates an H-bridge chip that allows the motor to reverse directional operation and generate a vacuum pressure for the filling operation.

Exemplary embodiments of the present disclosure also provide advantages in using an external fill device to fill the fluid delivery device with medicament whereby the external fill device axially displaces a main gear drive to disengage a drive train from a motor of the fluid delivery device and engage the leadscrew. This configuration allows the fluid delivery device to be filled with medicament faster and easier because less power and time is needed when the drive train is disengaged from the motor of the fluid delivery device. Such a configuration also allows the external fill device to not be constrained in size for motor and battery selection, for example. The fluid delivery device also advantageously includes a resealable septum to selectively provide fluid access and sealing before, during and after filling.

Exemplary embodiments of the present disclosure further provide an advantageous vial adapter that is configured to simultaneously engage a vial and an injection mechanism of a fluid delivery device. Specifically, the vial adapter advantageously provides an elastomer that is configured to secure to an outer diameter of a needle cannula of the injection mechanism of the fluid delivery device. Such a configuration avoids the need to integrate a filling mechanism into the fluid delivery device but rather provides a simple adapter for filling.

Furthermore, exemplary embodiments of the present disclosure provides an advantageous tabletop vial adapter that is configured to simultaneously engage a vial and a tabletop whereby the tabletop acts like a docking station and is configured to carry the fluid delivery device. This configuration provides another stable means to fill the fluid delivery device with medicament.

Aspects of illustrative embodiments provide a fluid delivery device comprising a reservoir configured to carry medicament, an insertion mechanism that delivers medicament to a patient, a vial connector system configured to engage and disengage a vial for filling of the medicament into the reservoir, and a fluid path establishing fluid communication between the reservoir and the insertion mechanism, wherein the vial connector system is disposed in the fluid path between the reservoir and the insertion mechanism.

Aspects of the illustrative embodiments further provide a system for filling and delivery of medicament, the system comprising a fluid delivery device comprising a reservoir configured to carry medicament, an insertion mechanism that delivers medicament to a patient, a leadscrew that drives the medicament to exit the reservoir, a main drive gear in a drive train, and a motor that cooperates with the drive train to axially move the leadscrew to dispense the medicament from the reservoir to the insertion mechanism through a fluid path, and an external fill device comprising a medicament cavity that carries the medicament, and an engagement member that is configured to engage the fluid delivery device, wherein when the engagement member engages the fluid delivery device, the engagement member axially displaces the main drive gear to disengage the drive train from the motor and engage the leadscrew for filling the fluid delivery device with the medicament.

Aspects of the illustrative embodiments also provide a vial adapter for engaging a vial carrying medicament and engaging a fluid delivery device, the vial adapter comprising a mounting surface configured to engage an insertion mechanism of the fluid delivery device, and an elastomer that is configured to engage an outer diameter of a cannula in the insertion mechanism to provide a scaling surface, wherein the vial adapter engages the fluid delivery device and the vial to fill the fluid delivery device with medicament while maintaining a sealed interface.

Aspects of the illustrative embodiments additionally provide a vial adapter for engaging a filling station, the vial adapter comprising an outer housing that is configured to engage a vial, an inner housing disposed in the outer housing, and a check valve to regulate air pressure, wherein when the vial adapter engages the filling station, the inner housing engages the outer housing to establish fluid communication.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims.

### BRIEF DESCRIPTION OF THE DRAWINGS

The above and/or other aspects and advantages of embodiments of the illustrative embodiments will be more readily appreciated from the following detailed description, taken in conjunction with the accompanying drawings, of which:

FIG. 1 is a side elevation view of a wearable fluid delivery device constructed in accordance with an exemplary embodiment;

FIG. 2 is a right perspective view of the fluid delivery device of FIG. 1 with the cover removed;

FIG. 3 is a block diagram of exemplary components of a fluid delivery device constructed in accordance with an exemplary embodiment;

FIG. 4 is a schematic of a vial connector system integrated into a fluid delivery device in accordance with an exemplary embodiment:

FIG. 5 is a schematic of the vial connector system of FIG. 4 engaged to a vial for filling the fluid delivery device;

FIG. 6 illustrates a transparent right perspective view of a durable external fill mechanism engaged to a fluid delivery device in accordance with an exemplary embodiment;

FIG. 7 illustrates a transparent section view of the durable external fill mechanism disengaged from the fluid delivery device of FIG. 6;

FIG. 8 illustrates a transparent section view of the durable external fill mechanism of FIG. 6 engaged to the fluid delivery device where a main drive gear is displaced;

FIG. 9 illustrates a transparent left perspective view of an outer cover of the fluid delivery device of FIG. 6 with a resealable connector;

FIG. 10 illustrates a cross sectional view of a vial adapter engaging an insertion mechanism of the fluid delivery device and a vial in accordance with an exemplary embodiment;

FIG. 11 illustrates a cross sectional view of a tabletop vial adapter engaged to a vial in accordance with an exemplary embodiment; and

FIG. 12 illustrates a perspective view of the tabletop vial adapter of FIG. 11 engaged to a tabletop carrying a fluid delivery device.

Throughout the drawing figures, like reference numbers will be understood to refer to like elements, features and structures.

### DETAILED DESCRIPTION OF ILLUSTRATIVE EMBODIMENTS

As will be appreciated by one skilled in the art, there are numerous ways of carrying out the examples, improvements, and arrangements of a pump in accordance with embodiments disclosed herein. Although reference will be made to the illustrative embodiments depicted in the drawings and the following descriptions, the embodiments disclosed herein are not meant to be exhaustive of the various alternative designs and embodiments that are encompassed by the disclosed technical solutions, and those skilled in the art will readily appreciate that various modifications may be made, and various combinations can be made with departing from the scope of the disclosed technical solutions. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. The specification describes specific examples to accomplish a more general goal that may be accomplished in another way.

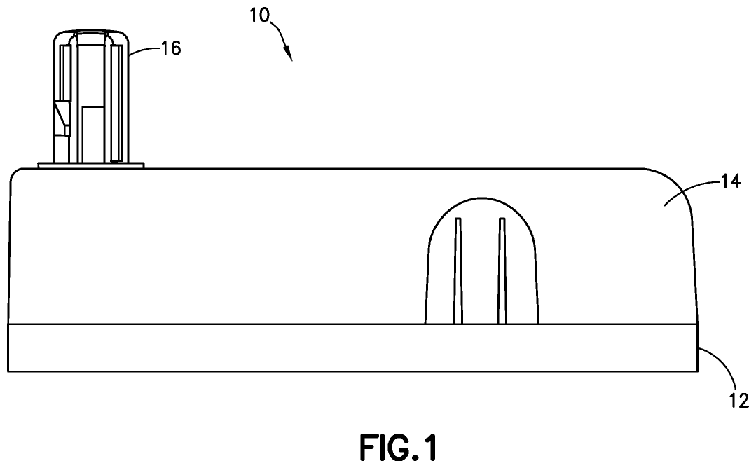

FIG. 1 is a side view of a wearable fluid delivery device 10 constructed in accordance with an exemplary embodiment. The fluid delivery device 10 comprises a baseplate 12, a cover 14, and an insertion mechanism 16 in an undeployed position (retracted position).

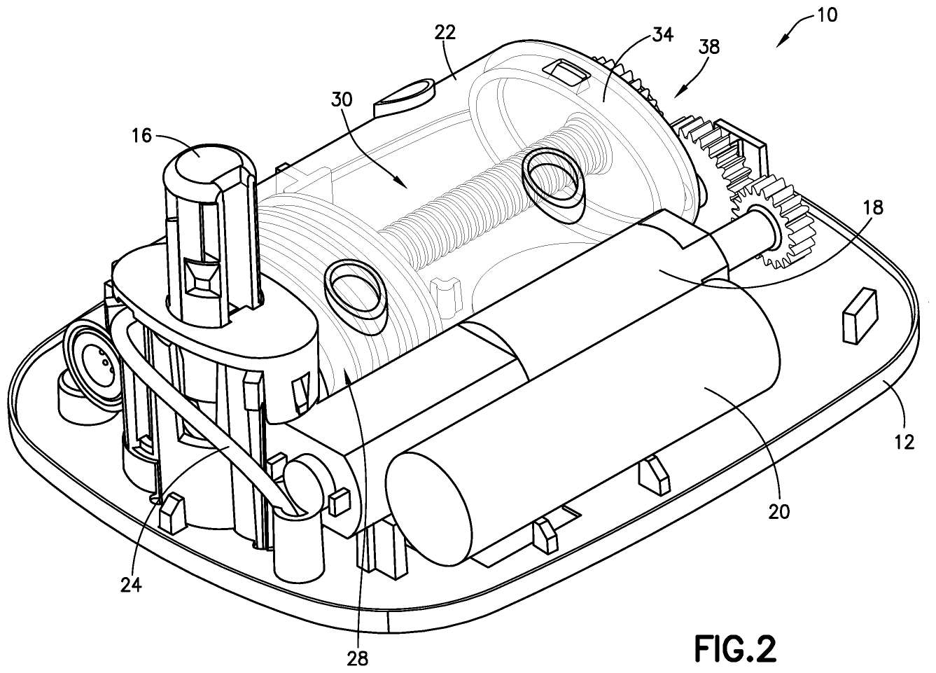

FIG. 2 is a perspective view of the fluid delivery device of FIG. 1 with the cover removed. The baseplate 12 supports the insertion mechanism 16, a motor 18, a power source such as a battery 20, a control board (not shown), and a reservoir 22 or container for storing a fluid to be delivered to a user via an outlet fluid path 24 from an outlet port of reservoir to the insertion mechanism 16. The reservoir 22 can also have an inlet port connected via an inlet fluid path 26 to a fill port (e.g., provided in the baseplate 12). The reservoir 22 contains a plunger 28 having a stopper assembly 28. The proximal end of the reservoir 22 is also provided with a plunger driver assembly 30 having a gear anchor 34, an outermost drive screw 36 that is rotated via a gear train 38 connected to the motor 18. Although a gear train 38 is shown for illustrative purposes, the drive mechanism can be gears, ratchets, or other methods of inducing rotation from a motor.

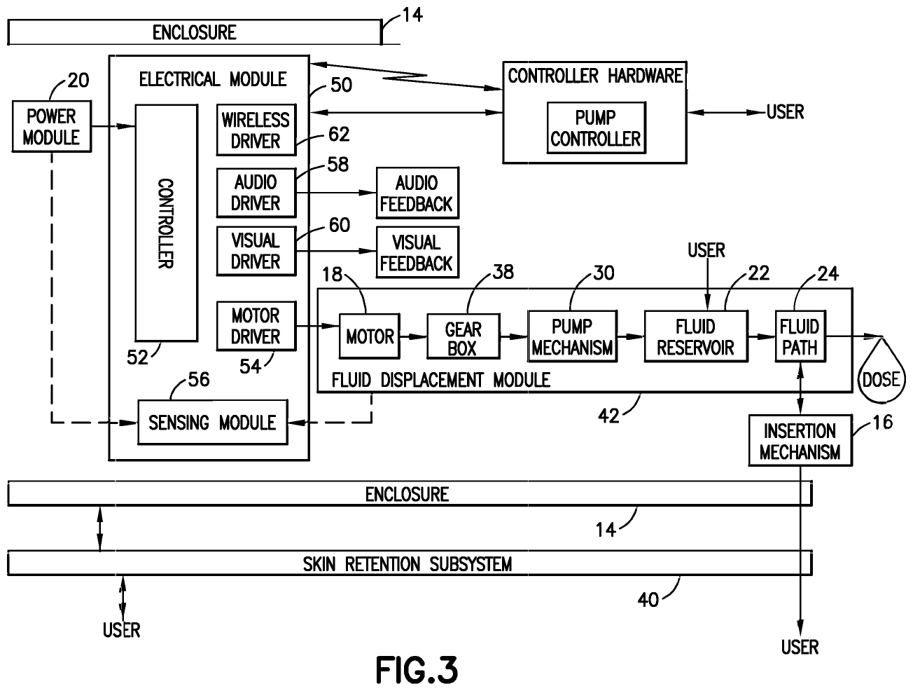

FIG. 3 is a block diagram of exemplary components of a fluid delivery device constructed in accordance with an exemplary embodiment. The cover/housing or device 10 housing is indicated at 14. The device 10 has skin retention subsystem 40 such as an adhesive pad to connect the device 10 to a user's skin. The fluid delivery device 10 further comprises the reservoir 22, the insertion mechanism 16, and a fluid displacement module 42 that can include the motor 18, gear train 38, pump mechanism (e.g., plunger driver assembly 30), and outlet path 24. The fluid delivery device further comprises electrical components such as a power module (e.g., battery 20), and an electrical module 50 comprising a controller 52, a motor driver 54 and other optional components.

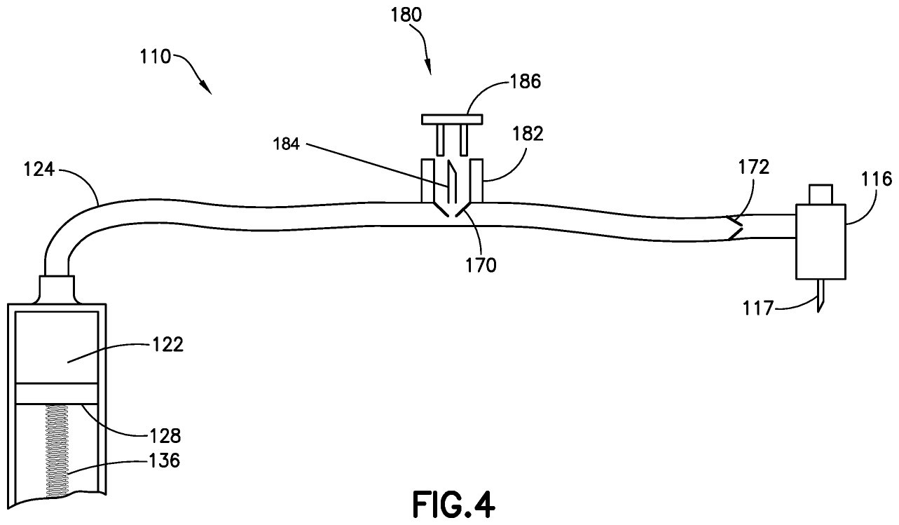

FIG. 4 is a schematic of a vial connector system 180 integrated into an exemplary fluid delivery device 110 according to one embodiment. The fluid delivery device 110 includes the components described above so long as these features do not contradict the features of this embodiment.

Specifically, the fluid delivery device 110 includes at least, a fluid path 124, a plunger head 128 attached to a plunger, a leadscrew 136 (also referred to as drive screw), a reservoir 122 and an insertion mechanism 116 comprising a patient needle 117.

The reservoir 122 is used to carry the medicament in the fluid delivery device 110. The reservoir 122 cooperates with a motor (as similarly illustrated above), a plunger head 128, a plunger (not shown) and a leadscrew 136 that moves axially to expel medicament from the reservoir 122 as commonly understood by one skilled in the art. During operation of the fluid delivery device 110, the fluid exits the reservoir 122 and travels to the insertion mechanism 116 and through the patient needle 117 for medicament delivery. The reservoir 122 and the insertion mechanism 116 are in fluid communication via the fluid path 124.

The motor of the fluid delivery device 110 advantageously includes an H bridge chip. The H bridge chip is commonly understood to be capable of reversing rotational operation and reversing output for a motor. The advantageous use of the H bridge chip overcomes the challenges with having a plunger that is fixed in place by a rigid connection and engaged to a lead screw that is unable to be back driven. Fluid delivery devices 110 such as infusion pumps of this style cannot be filled using traditional methods. The configuration of using a motor with an H bridge chip can create a vacuum in a closed system. This function is advantageously applied for use in the fluid delivery device 100, specifically during the filling operation because the filling operation functions in a naturally opposite manner from the medicament delivery operation. Thus, the plunger of the fluid delivery device 110 advantageously has a dual function of aspirating air and dispensing medicament.

The vial connector system 180 is disposed in the fluid path 124 between the reservoir 122 and the insertion mechanism 116. The vial connector system 180 is advantageously integrated into the fluid delivery device 110 for filling the reservoir 122 of the fluid delivery device 110. All tools used for filling are advantageously disposed in the fluid delivery device 110 such that no further devices or components are needed to transport and carry with the fluid delivery device 110. Moreover, this use of vial connection and flow direction control allow for the fluid delivery device 110 to accurately fill with higher accuracy than manual filling options.

The vial connector system 180 includes retaining features 182, a retractable needle 184 and a safety cap 186. The retaining features 182 include, for example, a snap fit or a mating shape for engaging an insulin bottle, for retaining the vial connector system 180 to a vial 102, although other means are contemplated by one skilled in the art. The design of the retaining features 182 may be adapted to optimize the connections to standardized vials 102. There may also be different attachments and variations for adapting to vials 102 of different sizes and shapes.

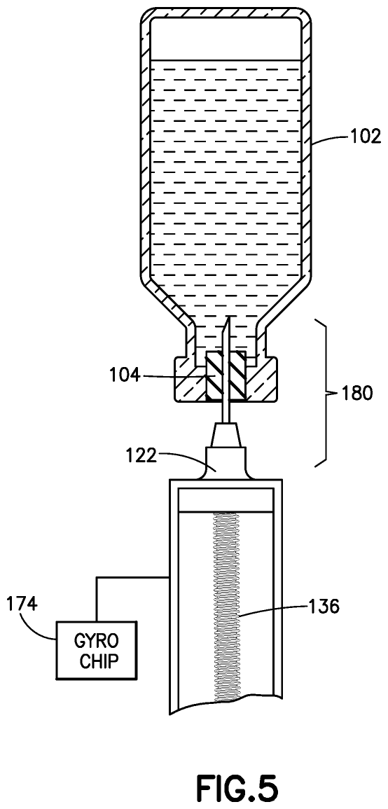

As illustrated in FIGS. 4 and 5, the retractable needle 184 is configured to pierce or open a septum 104 in the vial 102 to fill the reservoir 122 of the fluid delivery device 110 with medicament. The retractable needle 184 is retracted and shielded by the safety cap 186 when not in use to prevent inadvertent use.

In another embodiment, the vial connector system 180 dispenses air into the connected vial 102 to create a pressure gradient that assists in making the flow direction more favorable to filling into the reservoir 122. Specifically, the fluid delivery device 110 may be started in the retracted position, with a reservoir 122 full of air that can be dispensed into the vial 102 in order to generate the pressure gradient that assists in flow of the fluid to the reservoir 122.

The retractable needle 184 can either be retracted manually or automatically. Specifically, manual retraction is configured, for example, by snap fitting a housing carrying the needle to atop surface of fluid delivery device 110. The housing is spring loaded such that a user can disengage the snap fit between the housing and the top surface of the fluid delivery device 110. Upon disengagement, the retractable needle 184 moves to an extended position via the spring force for filling the fluid delivery device 110. After filling the fluid delivery device 110, the user pushes the housing and compressing the spring into the snap fit engagement with the top surface of the fluid delivery device to return to a retracted position.

Automatic retraction is configured, for example, by applying a mechanism used in a mechanically spring loaded pen. An exemplary button can be disposed on a bottom surface of the fluid delivery device to act similarly to a button on an automatically retracted pen. Exemplary examples of manual and automatic retraction are provided, although other means are contemplated by one skilled in the art.

The fluid delivery device 110 also advantageously includes two check valves 170, 172 that provide one way fluid flow during filling and during medicament delivery. Specifically, as illustrated in FIG. 4, the vial connector system 180 includes a check valve 170 and the insertion mechanism 116 includes a check valve 172. The check valve 170 of the vial connector system 180 prevents medicament from exiting the fluid delivery device 110 through the retractable needle 184. In this manner, the check valve 170 of the vial connector system 180 ensures that fluids can only exit through the insertion mechanism 116. The check valve 172 of the insertion mechanism 116 prevents fluid from entering the fluid delivery device 110 during the filling operation. In this manner, the check valve 172 of the insertion mechanism 116 ensures that fluid can only be filled and enter through the retractable needle 184 of the vial connector system 180.

FIG. 5 illustrates that the fluid delivery device 110 also includes a gyroscopic chip 174. The gyroscopic chip 174 is used to identify the orientation of the reservoir 122 of the fluid delivery device 110 and provide feedback to the user. It is important that the vial 102 is disposed above the fluid delivery device 110 to minimize air from entering into the fluid path 124. Also, the fluid delivery device 110 should be held upright for filling operation. Further, once the desired fill volume is taken into the reservoir 122, the fluid delivery device 110 is oriented using the gyroscopic chip 174 to ensure device orientation is conducive to expelling any retained air in the system.

When the fluid delivery device 110 is filled, a dead volume and a prime volume is typically present in the fluid path 124. As illustrated in FIG. 4, the dead volume is the volume in the fluid path 124 between the reservoir 122 and the vial connector system 180. The prime volume is the volume in the fluid path 124 between the vial connector system 180 and the insertion mechanism 116. Both these volumes must be primed with the aid of the gyroscopic chip 174. A factor of safety of 3, for example, can be relied upon to ensure air removal during priming. Specifically, the air volume can be determined based on fluid path dead volume calculations and can be primed out with a higher volume than anticipated to remove excess air with a factor of safety.

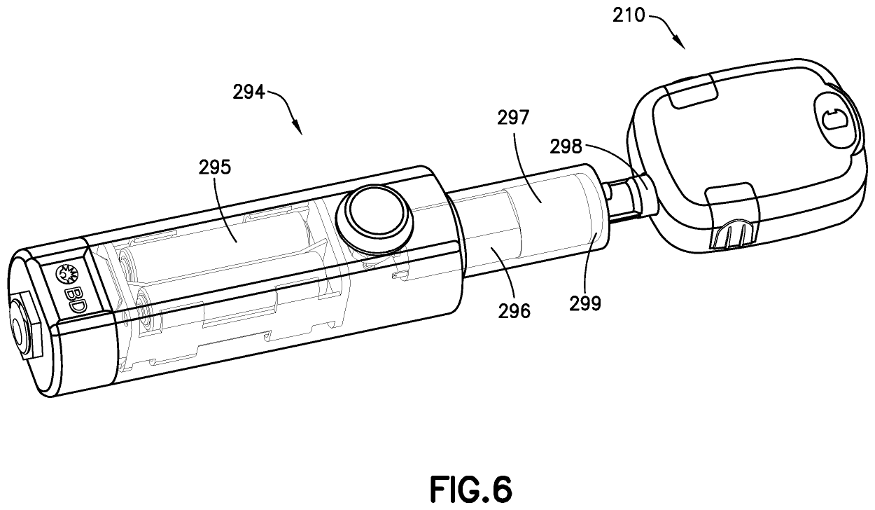

FIGS. 6-9 illustrate another embodiment including a fluid delivery device 210 such as an infusion pump or a patch pump as similarly described above and a durable external fill device 294 that replaces the integrated vial connector system 180 described in the embodiment above. The fluid delivery device 210 includes the components described above so long as these features are able to cooperate with the features of this embodiment.

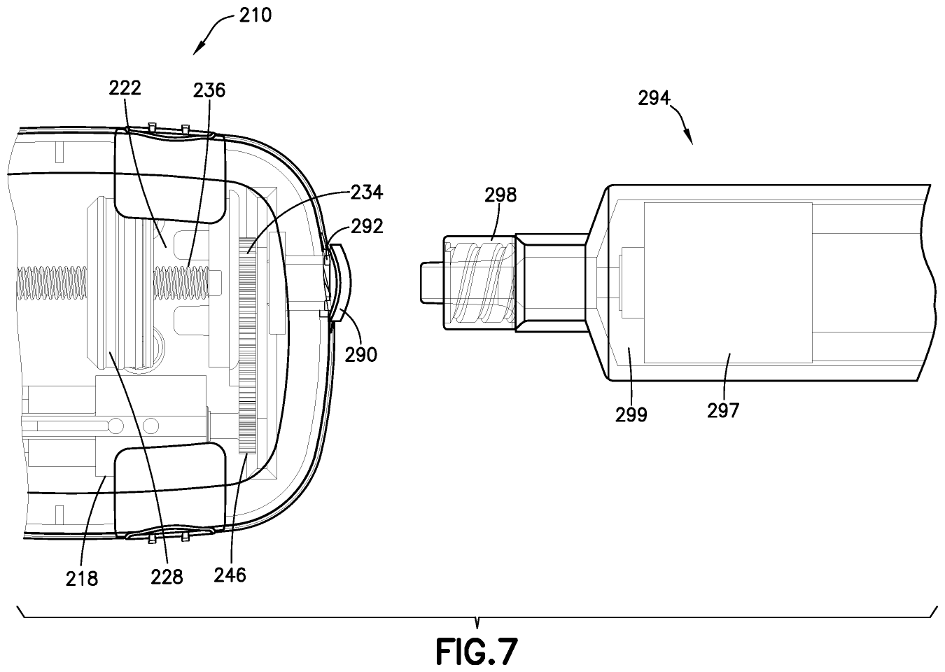

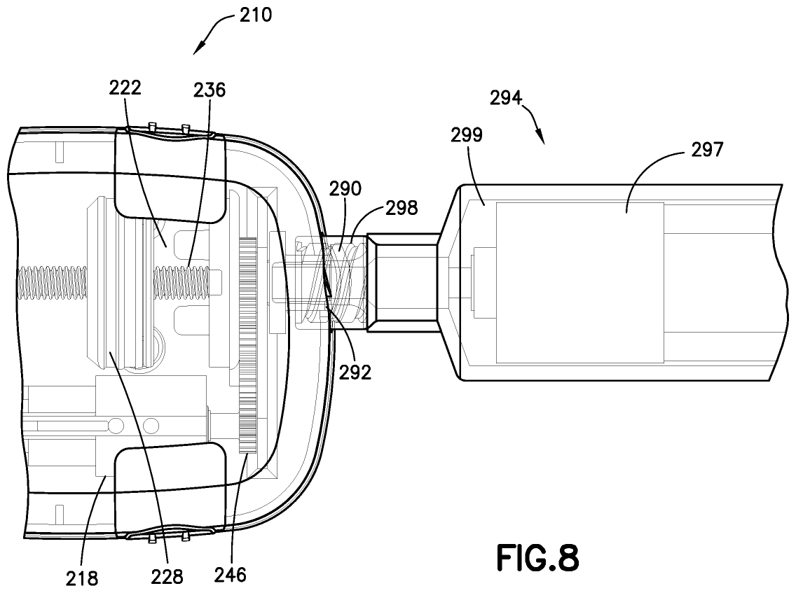

The fluid delivery device 210, as illustrated in FIGS. 7 and 8, includes a plunger head 228 connected to a plunger (not shown), a leadscrew 236 and a reservoir 222 as similarly described above. The fluid delivery device 210 further includes a pump motor 218 and a drive train 246 including a main drive gear 234 in a gearbox. The gears in the gearbox cooperate to engage the pump motor 218 and the leadscrew 236 to expel the fluid out of the reservoir 222.

The main drive gear 234 includes an inner diameter having a key slot engaging a protrusion in a gear drive shaft to provide axial movement. The main drive gear 234 also contacts a compression spring that forces the main drive gear 234 to engage the drive train 246 in an extended position. When axial pressure is applied to the main drive gear 234, the compression spring is compressed and the main drive gear 234 axially moves to disengage the drive train 246. Further operation of this configuration is described below.

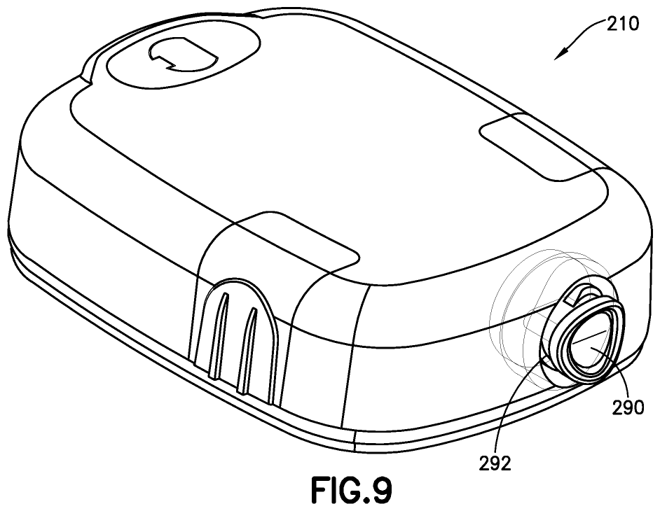

FIG. 9 illustrates that the fluid delivery device 210 also includes a resealable connector 290 comprising a split septum, and a luer connector 292. The resealable connector 290 provides selective opening and closing of the fluid delivery device 210 for sealing and for expelling medicament for medication delivery. An exemplary embodiment of the resealable connector 290 includes BD Q-Syte®. The luer connector 292 provides engagement to an external device such as the durable external fill device 294 as further described below.

FIGS. 6-8 illustrate details of the durable external fill device 294 which includes a power source 295, a motor 296, a plunger head 297 connected to a plunger, an engagement member 298 and a medicament cavity 299. The power source 295 includes batteries, for example, or any other suitable source of power as understood by one skilled in the art. The durable external fill device 294 can be advantageously designed to any size and shape to accommodate the power source 295 to mitigate power consumption risks.

The motor 296 is provided to drive the plunger head 297 via the plunger to dispense the medicament from the medicament cavity 299 for filling the fluid delivery device 210. The durable external fill device 294 can also be advantageously designed to any size and shape to accommodate the motor 296. Accordingly, a large motor capable of high torque can be used to move the fluid delivery device 210 or a fixed plunger of a syringe pump, for example. The engagement member 298 comprises a luer connector that engages the luer connector 292 of the fluid delivery device 210 as further described below.

Specifically, FIGS. 6 and 8 illustrate the durable external fill device 294 engaged to the fluid delivery device 210, while FIG. 7 illustrates the durable external fill device 294 disengaged from the fluid delivery device 210. When the durable external fill device 294 is engaged to the fluid delivery device 210, the plunger head 228 connected to a fixed plunger axially moves to a desired fill position. A fixed plunger design or rigid attachment of the plunger means that there are no moving parts (rotational or off axis movement) except for axial movement of the plunger connected to a plunger head and leadscrew.

FIG. 8 specifically shows that when the durable external fill device 294 is engaged to the fluid delivery device 210, the main drive gear 234 of the fluid delivery device 210 is axially displaced such that the compression spring is compressed and main drive gear 234 is disengaged from the drive train 246 as similarly described above. In this manner, the pump motor 218 is unable to operate the fluid delivery device 210.

Instead, the motor 296 of the durable external fill device 294 is able to connect to the leadscrew 236 of the fluid delivery device 210 via the engagement member 298. When the luer connector of the engagement member 298 is rotated halfway, the leadscrew 236 is engaged. When the luer connector of the engagement member 298 is engaged fully, an end button of the durable external fill device 294 is on top as illustrated in FIG. 6 and the main gear 234 is displaced as described above. Accordingly, the engagement member 298 of the durable external fill device 294 is advantageously able to back drive the leadscrew 236 of the fluid delivery device 210 to fill the reservoir 222 with the medicament. Such a configuration provides several advantages as described below.

The disengaged gear train 246 in the fluid delivery device 210 advantageously provides for a time efficient and energy efficient means to fill the fluid delivery device 210. Otherwise, overcoming the gear train 246 to fill the fluid delivery device 210 would be time consuming and take much more energy based on the gear ratio and the resulting amplified torque provided by the gear train 246. Further, engaging and disengaging the main drive gear 234 provides a simple solution for improving the filling operation. Furthermore, the luer connection between the engagement member 298 of the durable external fill device 294 and the luer connector 292 in the fluid delivery device 210 provides a defined engagement and a defined axial displacement of the main drive gear 234. Accordingly, a user is assured that the durable external fill device 294 is positioned to fill the fluid delivery device 210 upon engagement.

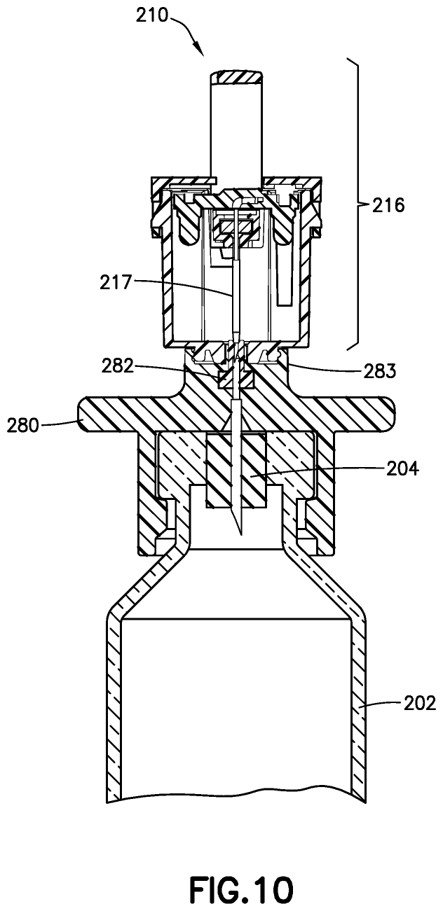

FIG. 10 illustrates another embodiment of a vial adapter 280 that is configured to engage a vial 202 having a septum 204 and an insertion mechanism 216 of the fluid delivery device 210. Specifically, a distal end of the vial adapter 280 includes a cavity configured to engage the vial 202. As similarly described above, other forms of engagement as understood by one skilled in the art such as a snap fit are contemplated herein. The distal end of the vial adapter 280 opens into a cavity whereby a needle is disposed. The needle of the vial adapter 280 engages the septum 204 of the vial 202 when the vial adapter 280 is engaged to the vial 202.

When filling the fluid delivery device 210, the vial 202 needs to be pressurized with positive pressure while the plunger in the fluid delivery device 210 is retracting. Alternately, the vial 202 needs to be vented in order to allow air to enter the vial 202 as fluid moves out so that vacuum pressure is not generated. One of these conditions should be present for proper filling of the fluid delivery device 210.

A proximal end of the vial adapter 280 includes an elastomer 282 and a mounting surface 283. The mounting surface 283 includes threads, for example, although other forms of engagement as understood by one skilled in the art are contemplated herein. The mounting surface 283 is configured to advantageously engage an insertion mechanism 216 of the fluid delivery device 210.

The elastomer 282 is press fit into an inner diameter of the vial adapter 280 and onto an outer diameter of the patient needle 217 of the insertion mechanism 216. In this configuration, a scaled interface is advantageously provided when the fluid delivery device 210 is filled with medicament from the vial 202 via the vial adapter 280.

The vial adapter 280 provides several advantage as described below. The vial adapter 280 is able to fill the fluid delivery device 210 with medicament without advantageously creating a separate filling mechanism. The fluid delivery device 210 does not require a fill septum or a fill port, for example. Instead, the insertion mechanism 216 typically used for medicament delivery is also used for filling. Such a configuration is simple and convenient. Further, the engagement between the vial adapter 280 and the fluid delivery device 210 provides a secure attachment and a sealed surface for medicament filling.

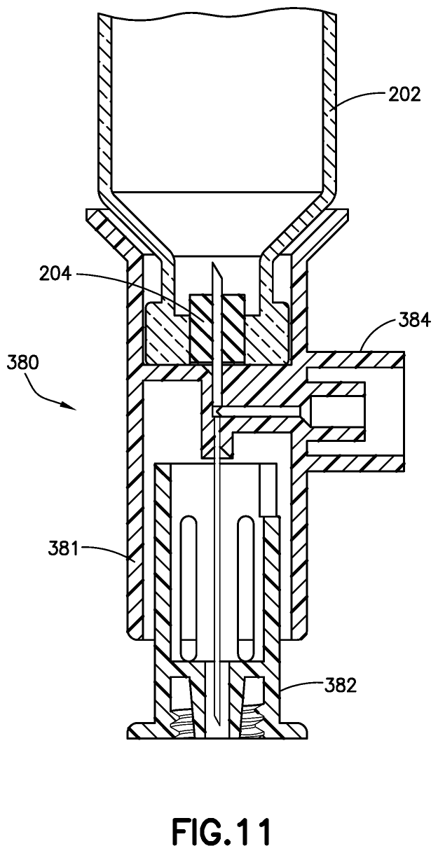

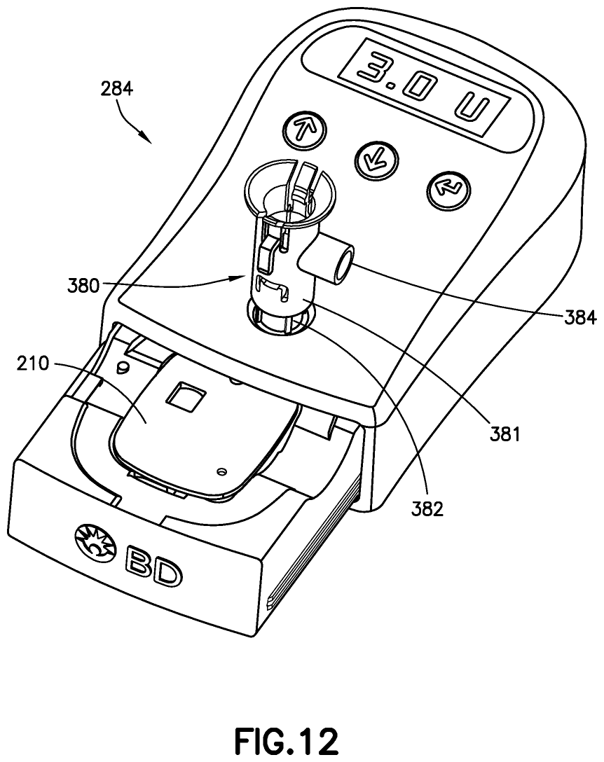

FIGS. 11 and 12 illustrate a tabletop vial adapter 380 configured to engage a tabletop filing station 284 also known as a handheld filling station. The tabletop filling station 284 is configured to carry the fluid delivery device 210 for filling with medicament. The tabletop filling station 284 can be placed on a surface of a table, secured to the table by screws, for example, or be held by a user to conveniently engage and fill the fluid delivery device 210 reliably.

The tabletop vial adapter 380 includes an outer housing 381, an inner housing 382 and a check valve 384. The tabletop vial adapter 380 components are not durable in the same way as the durable external fill device 294 cooperating with the resealable connector 290 of the fluid delivery device 210 as described above. Specifically, the resealable connector 290 does not contact the medicament, such as insulin. On the other hand, the tabletop vial adapter 380 contacts the medicament. Accordingly, the tabletop vial adapter 380 is required to be disposed of after use. The resealable connector 290 is built into a fluid delivery device 210 that is disposed after use. A proximal end of the outer housing 381 is configured to engage the vial 202. Specifically, the proximal end of the outer housing 381 includes a cavity surrounding a needle. The needle of the tabletop vial adapter 380 is configured to pierce the septum 204 of the vial 202 to establish fluid communication.

The outer housing 381 further includes a check valve 384. The check valve 384 comprises a one-way valve that advantageously regulates air pressure in the tabletop vial adapter 380. Specifically, the check valve 384 releases excess air from the tabletop vial adapter 380 and excess air from the fluid delivery device 210. The check valve 384 also prevents the formation of a vacuum in the tabletop vial adapter 380 and the fluid delivery device 210.

The outer housing 381 is configured to carry the inner housing 382. The inner housing 382 is configured to engage the tabletop vial adapter 380. Specifically, FIG. 12 illustrates the inner housing 382 engaged to the tabletop filling station 284 and the fluid delivery device 210 installed in the tabletop filling station 284. When the inner housing 382 engages the tabletop filling station 284, the inner housing 382 snap fits into engagement with the outer housing 381. This engagement also causes a distal end of the needle of the tabletop vial adapter 380 to fluidly communicate with the fluid delivery device 210.

In another embodiment, the vial connection (or inner housing 382) can also be attached to one of various portions of the fluid path of the fluid delivery device 210 or at the insertion mechanism (as understood in FIG. 12), designated as a filling site. In this configuration, the fluid delivery device 210 would be configured with an attachment mechanism that engages a distal end of the inner housing 382.

The tabletop filling station 284 includes a drawer that opens and closes. The drawer opens so that the fluid delivery device 210 can be inserted and automatically installed into the tabletop filling station 284 for filing. The fluid delivery device 210 is automatically aligned with a hole in the tabletop filling station 284 when the drawer is closed so that the fluid delivery device 210 can be filled when connected to the tabletop vial adapter 380.

The tabletop filling station 284 can be configured, for example, to only engage the tabletop vial adapter 380 when the drawer is closed and when the fluid delivery device 210 is present in the tabletop filling station 284. The tabletop filling station 284 can also be configured to operate via a motor only when the drawer is closed. The configured conditions described above can reduce the risk of the user misusing the tools or components as described above to fill the fluid delivery device 210.

Although various persons, including, but not limited to, a patient or a healthcare professional, can operate or use illustrative embodiments of the present disclosure, for brevity an operator or user will be referred to as a “user” hereinafter.

Although various fluids can be employed in illustrative embodiments of the present disclosure, for brevity the liquid in an injection device will be referred to as “fluid” hereinafter.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Further, terms such as up, down, bottom, and top are relative, and are employed to aid illustration, but are not limiting. Terms of degree, such as “substantially” or “approximately” are understood by those of ordinary skill to refer to reasonable ranges outside of the given value, for example, general tolerances associated with manufacturing, assembly, and use of the described embodiments. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

What is claimed is:

1. A fluid delivery device comprising:
a reservoir configured to carry medicament;
an insertion mechanism that delivers medicament to a patient;
a vial connector system configured to engage and disengage a vial for filling of the medicament into the reservoir; and
a fluid path establishing fluid communication between the reservoir and the insertion mechanism; wherein
the vial connector system is disposed in the fluid path between the reservoir and the insertion mechanism.

2. The fluid delivery device of claim 1, wherein the vial connector system includes retaining features for mating with the vial.

3. The fluid delivery device of claim 1, wherein the vial connector system includes a retractable needle for filling the reservoir.

4. The fluid delivery device of claim 3, wherein the vial connector system includes a safety cap for shielding the retractable needle.

5. The fluid delivery device of claim 3, wherein the retractable needle retracts manually.

6. The fluid delivery device of claim 3, wherein the retractable needle retracts automatically.

7. The fluid delivery device of claim 1, wherein the vial connector system includes a check valve to prevent the vial connector system from dispensing the medicament.

8. The fluid delivery device of claim 1, wherein the insertion mechanism includes a check valve to prevent the insertion mechanism from filling the reservoir.

9. The fluid delivery device of claim 1, further comprising at least two check valves in the fluid path to control the medicament flow during the filling and during operation.

10. The fluid delivery device of claim 1, further comprising a gyroscopic chip to provide orientation feedback of the fluid delivery device during the filling operation.

11. The fluid delivery device of claim 1, further comprising a motor including an H-bridge chip that allows the motor to change direction and generate a vacuum pressure for the filling operation.

12. A system for filling and delivery of medicament, the system comprising:
a fluid delivery device comprising:
a reservoir configured to carry medicament;
an insertion mechanism that delivers medicament to a patient;
a leadscrew that causes the medicament to exit the reservoir;
a main drive gear in a drive train; and
a motor that cooperates with the drive train to axially move the leadscrew to dispense the medicament from the reservoir to the insertion mechanism through a fluid path; and
an external fill device comprising:
a medicament cavity that carries the medicament, and
an engagement member that is configured to engage the fluid delivery device; wherein
when the engagement member engages the fluid delivery device, the engagement member axially displaces the main drive gear to disengage the drive train from the motor and engage the leadscrew for filling the fluid delivery device with the medicament.

13. The system of claim 12, wherein when the engagement member disengages the fluid delivery device, the main drive gear is engaged to the drive train so that the motor can drive the leadscrew for medication delivery.

14. The system of claim 12, wherein the fluid delivery device further comprises a resealable connector including a split septum to provide selective sealing during engagement and disengagement of the engagement member and the fluid delivery device.

15. The system of claim 12, wherein the engagement member comprises a luer connector.

16. A vial adapter for engaging a vial carrying medicament and engaging a fluid delivery device, the vial adapter comprising:
a mounting surface configured to engage an insertion mechanism of the fluid delivery device, and
an elastomer that is configured to engage an outer diameter of a cannula in the insertion mechanism to provide a sealing surface; wherein
the vial adapter engages the fluid delivery device and the vial to fill the fluid delivery device with medicament while maintaining a sealed interface.

17. The vial adapter of claim 16, wherein when the vial adapter fills the fluid delivery device, the fluid delivery device comprises a patch pump.

18. A vial adapter for engaging a filling station, the vial adapter comprising:
an outer housing that is configured to engage a vial;
an inner housing disposed in the outer housing; and
a check valve to regulate air pressure; wherein
when the vial adapter engages the filling station, the inner housing engages the outer housing to establish fluid communication.

19. The vial adapter of claim 18, wherein when the vial adapter is engaged to the vial and the filling station and when a fluid delivery device is disposed in the filling station, the fluid delivery device is filled with the medicament.
